(19) United States
(12) Patent Application Publication
(10) Pub. No.: US 2026/0047921 A1
(43) Pub. Date: Feb. 19, 2026

(54) DEVICES FOR HOLDING INTRAOCULAR LENS ALONG AN OPTICAL AXIS THEREOF

(71) Applicant: Eyemed Technologies Ltd, KIRYAT ONO (IL)

(72) Inventors: Vadim SHMUKLER, Rishon LeZion (IL); Nir ISRAELI, Ramat Gan (IL); Philip MITSEL, Lod (IL)

(21) Appl. No.: 19/101,757

(22) PCT Filed: Sep. 10, 2023
(86) PCT No.: PCT/IL2023/050972

§ 371 Date: Feb. 6, 2025

(30) Foreign Application Priority Data
Sep. 12, 2022 (IL) 296395

(51) Int. Cl. A61F 2/16

(52) U.S. Cl. CPC A61F 2/16; A61F 2002/16902; A61F 2210/0038; A61F 2220/0091

(57) ABSTRACT

Devices configured to be implanted in a lens capsule of a human eye and securely hold an intraocular lens (IOL), and operable to displace the IOL relative to an optical axis of the IOL; the devices comprising a first member configured to be fixedly positioned inside the lens capsule, a second member to which the IOL is fixedly attachable, and a bendable structure attached at a first end thereof to the first member and at a second end thereof to the second member; the bendable structure being configured to be remotely controllably bended at various bending levels to thereby vary location of a periphery of the IOL, when the IOL is attached to the second member, along the IOL optical axis.

### TECHNOLOGICAL FIELD

The present invention is in the field of medical devices, and relates specifically to devices configured for holding intraocular lenses in vivo.

### BACKGROUND

Various medical conditions related to vision and eyesight are treated by replacement of the natural eye lens with an artificial intraocular lens (IOL). Eye-related problems such as cataract, eye trauma, vision refractive errors including far-sightedness (hyperopia), near-sightedness (myopia) and astigmatism can be solved by IOL replacement surgery. This treatment can be beneficial in other eye conditions in people who are not eligible for laser treatment.

Cataracts are the most prevalent ocular disease worldwide, being the cause of half of blindness and third of visual impairment in the world. About twenty-five million patients worldwide undergo cataract surgery on annual basis.

The typically implanted IOL provides selected focal length and optical power that should allow the patient to have a fairly good vision. However, it is often difficult to predict the exact characteristics of the lens necessary to correct the impaired vision. For example, currently, less than 50% of patients achieve their targeted vision after treatment, even with state-of-the-art multi-focal and other presbyopia correcting intraocular lenses, resulting in that post-surgery patients should often wear glasses for reading or distance vision.

Although it is a frequent surgical procedure, IOL replacement surgery involves several challenges, for example: prediction of exact lens characteristics (ELP); lens-positioning error during the surgery; tilt or shift after the surgery and during eye healing process; and change of the corneal cylinder in the elderly. There are a few types of IOLs used to correct visual impairment, such as Mono-focal, Multi-focal and Toric (with possible combinations in the same lens). Following the installation and healing process, all kinds of IOL may move and deviate from the designed optical axis, hence requiring compensation by optimizing the IOL location inside the lens capsule. Modification of the IOL location may be needed around the optical axis of the IOL, to correct for astigmatism issues, or along the optical axis of the IOL, to correct focusing problems. There are several techniques, both invasive and non-invasive, used to implement the compensations, such as repeated surgery to displace the IOL; use of a unique UV sensitive polymer that enables compensation by post deforming of the lens; and/or modification of the IOL shape by use of laser radiation.

### GENERAL DESCRIPTION

The present invention provides techniques for post adjustment and optimization of position of an intraocular lens (IOL) that has been already implanted inside the lens capsule. By IOL, the applicants refer basically herein to the optical lens itself which does not include the cradle that holds the IOL.

The described techniques, systems and devices enable non-invasive, remote, and repeated corrections of the IOL position and/or orientation (tilt) relative to the optical axis of the lens, enabling the procedure to be performed relatively easily and in short time, e.g. in the clinic, while eliminating the need for additional invasive surgical procedures.

The invention provides IOL holding/supporting systems/devices (e.g. in the form of a cradle) that include/integrate a movement system/mechanism/assembly operable to displace and adjust the position/orientation of the IOL along its optical axis. The movement system/mechanism/assembly is configured to be activated remotely from outside the eye and apply correction of the IOL position in the optical axial direction (also called herein the axial or Z direction), thereby displacing/changing at least the location of the focal point of IOL along the Z direction.

The invention enables doctors to precisely adjust the position of the IOL based on the exact amount of visual correction needed to achieve the desired vision. The systems/devices disclosed herein are miniature enabling remote access to the integrated movement system/mechanism/assembly through the pupil of the eye. The diameter of the pupil extends between about 2 mm to about 4 mm in light conditions and between about 4 mm to about 8 mm in dark conditions. Accordingly, the described systems/devices allow for accommodating an IOL having a diameter in the range of about 3.5-6mm. The technique of the present invention enables achieving tiny axial step distances and/or tiny tilt angles in the desired range(s).

Additionally, the described systems and devices are elastic and foldable, at least in a working range of temperature, hence facilitating insertion and implantation in the eye capsule.

Thus, according to one aspect, there is provided a device configured to be implanted in a lens capsule of a human eye and securely hold an intraocular lens (IOL), and operable to displace the IOL relative to an optical axis of the IOL, the device comprising:
a first member configured to be fixedly positioned inside the lens capsule;
a second member to which the IOL is fixedly attachable; and
a bendable structure attached at a first end thereof to the first member and at a second end thereof to the second member; the bendable structure being configured to be remotely controllably bended at various bending levels to thereby vary location of a periphery of the IOL, when the IOL is attached to the second member, along the IOL optical axis.

In some embodiments, the device comprises a locking mechanism configured to be remotely controllable and operable to at least partially lock or release one or more portions of the bendable structure to/from either the first member or the second member, to thereby vary the location of the periphery of the IOL, when the IOL is attached to the second member, along the IOL optical axis. The locking mechanism can be operable to at least partially lock or release the one or more portions of the bendable structure to/from either the first member or the second member, in a way such that first and second planes defined by the first and second members respectively remain parallel therebetween and perpendicular to the IOL optical axis during varying the location of the periphery of the IOL along the IOL optical axis.

In some embodiments, the locking mechanism is operable to at least partially lock or release the one or more portions of the bendable structure to/from either the first member or the second member, in a way such that a first plane defined by the first member becomes tilted with respect to a second plane defined by the second member during varying the location of the periphery of the IOL along the IOL optical axis.

In some embodiments, the locking mechanism comprises a plurality of remotely controllable lockers, each of the lockers being configured to lock and release a portion of the bendable structure to/from either the first or second members.

In some embodiments, the bendable structure comprises a plurality of elongated bendable members, each of the elongated bendable members having a first end permanently attached to the first member and a second end permanently attached to the second member.

In some embodiments, the locking mechanism comprises at least two groups of remotely controllable lockers and said bendable structure comprises respective at least two elongated bendable members, each of the groups of controllable lockers being operable to lock or release the respective elongated bendable member to/from either the first or second members, each of the groups of controllable lockers comprises a plurality of controllable lockers arranged in a locker array such that sequential activation of the lockers in the locker array locks or releases respective portions of the elongated bendable member.

Symmetrical activation of the individual controllable lockers belonging to the different groups of the controllable lockers causes varying the location of the periphery of the IOL in a perpendicular direction to the IOL optical axis. Asymmetrical activation of the individual controllable lockers belonging to the different groups of the controllable lockers causes varying the location of the periphery of the IOL in a tilted direction to the IOL optical axis.

In some embodiments, the locking mechanism comprises a third member adjacent to the first member such that the bendable structure is enclosed between the first and third members, rotating the third member with respect to the first member locks or releases the one or more portions of the bendable structure to/from the first member, and varies the location of the peripheral of the IOL along the IOL optical axis.

In some embodiments, the bendable structure comprises a plurality of elongated bendable members, at least some of the elongated bendable members having a first end permanently attached to the first member and at least some of the elongated members having a second end permanently attached to the second member, each one of the plurality of elongated bendable members is twistable around a longitudinal axis thereof, the elongated bendable members being arranged in one or more pairs having the two elongated bendable members of the pair located on opposite sides with respect to the second member, remotely controlling the twist degree of the two elongated bendable members of the pair causes tilting the IOL, when attached to the second member, around an axis passing through the two elongated bendable members of the pair.

In some embodiments, the device is configured and operable to displace the IOL, when attached to the second member, in at least one of anterior or posterior direction relative to the first member.

In some embodiments, the device is configured and operable to displace the IOL, when attached to the second member, at least partially in a reversible manner.

In some embodiments, the bendable structure is made from a shape memory material treated such that default shape of the bendable structure is in its fully open state, wherein said locking mechanism is operable to vary position of the bendable structure between a fully closed state and the fully open state.

In some embodiments, each of the controllable lockers is made from a shape memory material treated such that at body temperature the locker is in a closed state locking the respective portion of the bendable structure and such that when heated to a predefined temperature, by absorbing energy from a remote energy source, the locker transitions into open state releasing the respective portion of the bendable structure. The bendable structure can be made from a shape memory material treated such that at body temperature a default shape of each elongated bendable member is in its fully twisted state and such that when heated to a predefined temperature, by absorbing energy from a remote energy source, the elongated bendable member transitions into a less twisted state. In some embodiments, the shape memory material comprises nitinol.

In some embodiments, the device is foldable such that it can be passed through a cross-section of about 2.54 mm² or about 1.8 mm circular diameter.

In some embodiments, the lockers are configured to be mechanically attachable to corresponding points on the first or second members. In some embodiments, the lockers are attachable mechanically to the first or second members by clipping or male-female setup. In some embodiments, the mechanical attachment is reversible.

In some embodiments, the bendable members include, at a side thereof facing the first or second member (as the case may be), sequential grooves and protrusions matching sequential protrusions and grooves at the opposite side of the first or second member, the lockers being aligned in their locking state with the protrusions of the bendable members and in their unlocking state with the protrusions of the first or second members.

According to another aspect, there is provided an IOL adjustment system comprising:
Any of the devices described above; and
a remote energy source configured and operable to provide said energy to heat one or more portions of the device.

In some embodiments, the remote energy source comprises a radiating element.

In some embodiments, the remote energy source comprises a laser source.

In some embodiments, the laser source is configured and operable to provide continuous laser radiation.

In some embodiments, the laser source is configured as an Argon laser source operable to provide light of a green spectrum.

In some embodiments, the laser source is configured and operable to provide laser power between 0.1-5 watt, and laser pulse width between 200-1000 ms.

In some embodiments, the remote energy source comprises an electromagnetic radiation transmitter and said one or more portions of the device comprise respective electromagnetic radiation receivers.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1L illustrate a first non-limiting example of a device configured to hold an IOL and operable to remotely adjust position of the IOL after it has been implanted inside an eye, in accordance with the invention;

FIGS. 2A-2E illustrate a second non-limiting example of a device configured to hold an IOL and operable to remotely adjust position of the IOL after it has been implanted inside an eye, in accordance with the invention;

FIG. 2AA-2AD illustrate a third non-limiting example of a device configured to hold an IOL and operable to remotely adjust position of the IOL after it has been implanted inside an eye, in accordance with the invention;

FIGS. 3A-3C illustrate a fourth non-limiting example of a device configured to hold an IOL and operable to remotely adjust position of the IOL after it has been implanted inside an eye, in accordance with the invention;

FIGS. 4A-4C illustrate a fifth non-limiting example of a device configured to hold an IOL and operable to remotely adjust position of the IOL after it has been implanted inside an eye, in accordance with the invention; and

FIGS. 5A-5D illustrate non-limiting examples of devices configured to protect the functional parts of any one of the above-described devices, in accordance with the invention.

### DETAILED DESCRIPTION OF EMBODIMENTS

The present invention is aimed at providing intraocular lens (IOL) holding devices that enable remote, non-invasive, and controlled post-adjustment of the position of the implanted lens with respect to the IOL optical axis.

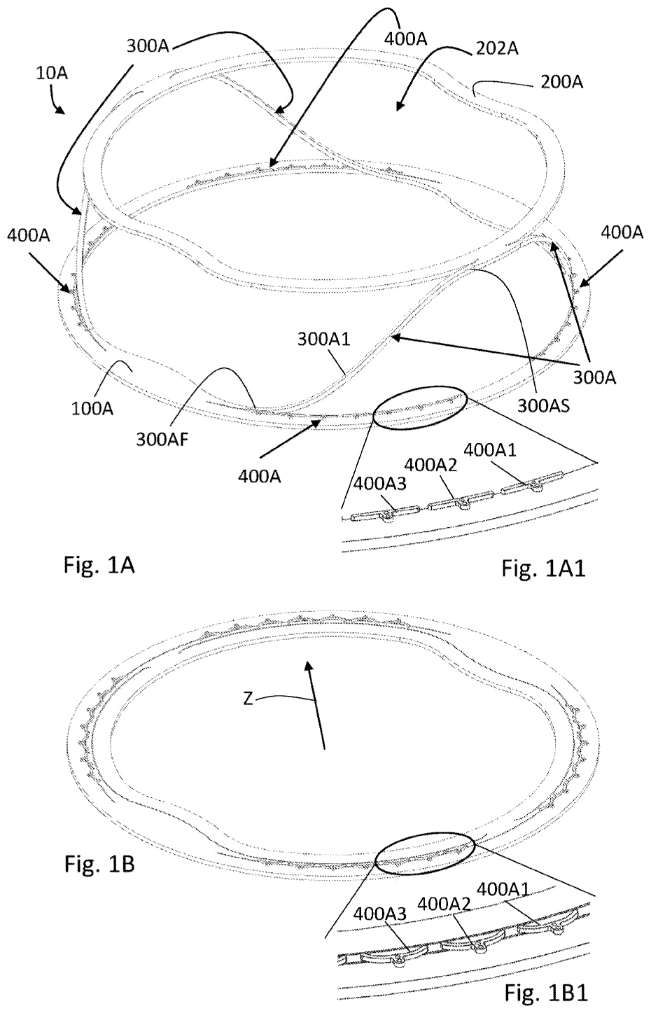

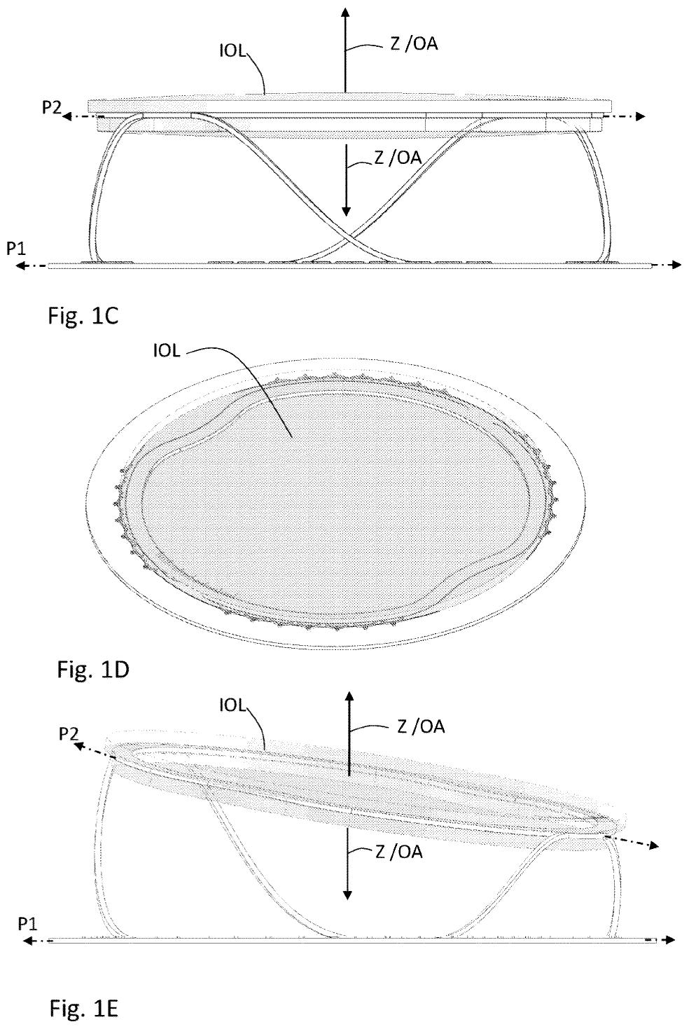

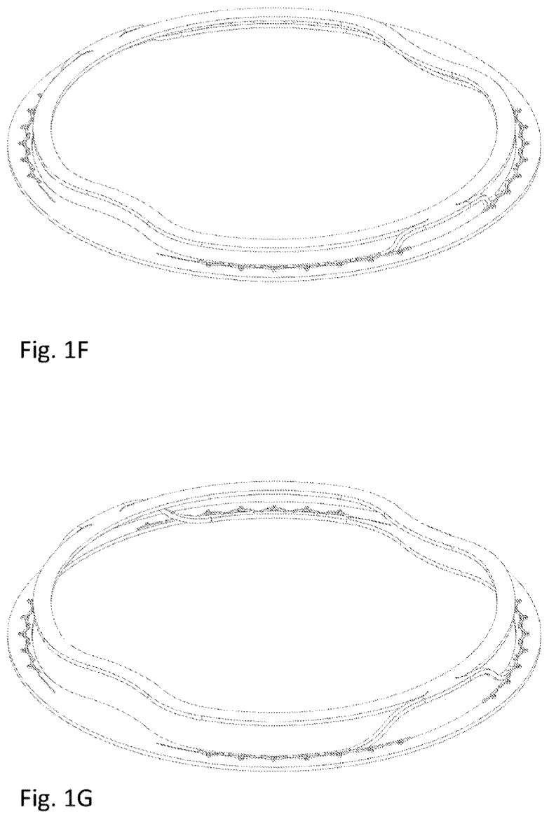

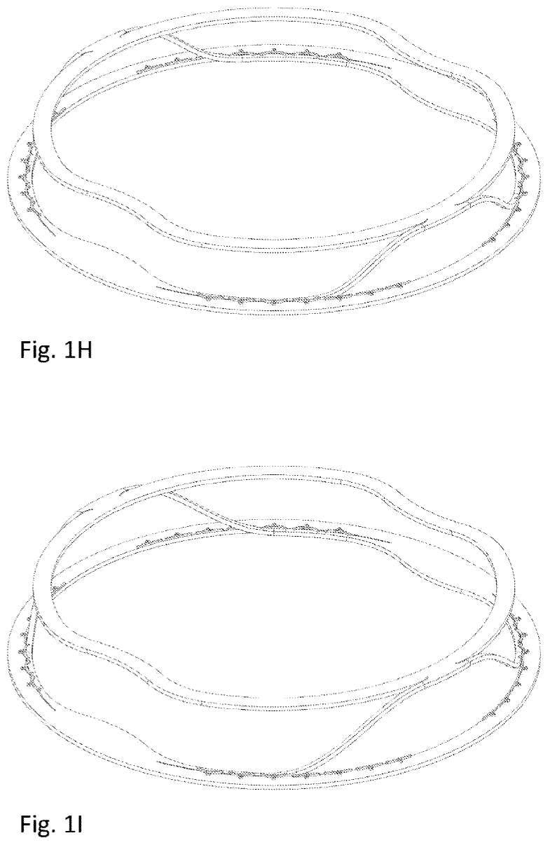

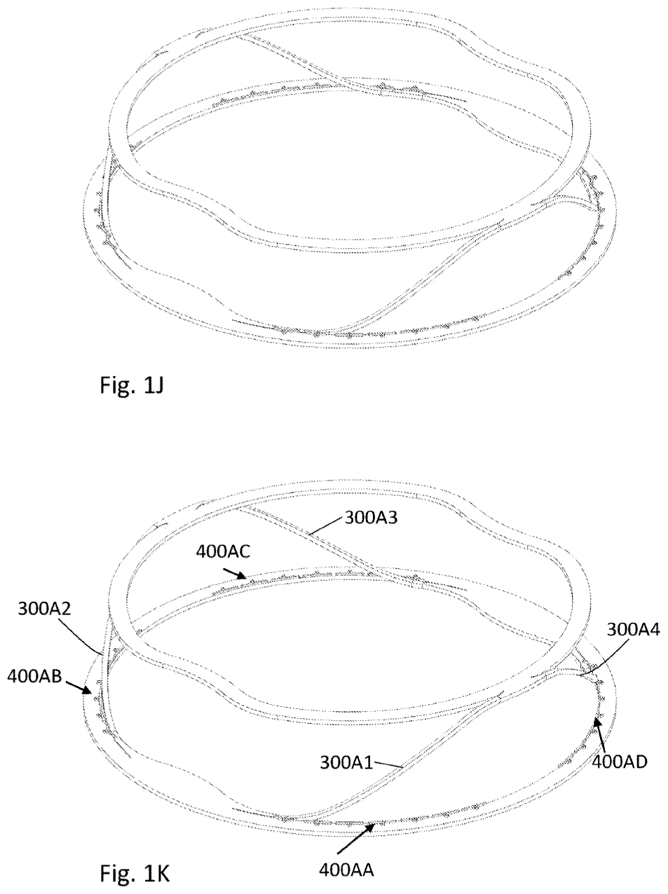

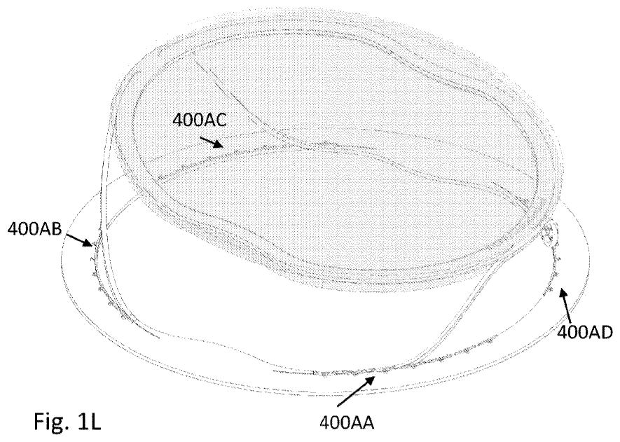

Reference is made to FIGS. 1A-1L, schematically illustrating a first non-limiting example of a device 10A, incorporating the principles of the technique of the present invention, the device 10A being configured to be implanted in a lens capsule of a human eye, securely hold an intraocular lens IOL and operable to displace at least a portion of the IOL along the IOL optical axis, by absorbing energy from a remote energy source (which is not part of the device 10A and can be selected from a variety of suitable energy sources, such as some laser sources, ultrasound sources, as will be further described below).

FIG. 1A is an isometric view of the device 10A in an open state, with maximal displacement of the IOL holding portion along the optical axis (OA)/Z direction; FIG. 1B is an isometric view of the device 10A in a closed state with zero displacement of the IOL holding portion along the optical axis/Z direction; FIG. 1A1 and 1B1 are close-up views of functional portions of the device 10A; FIGS. 1C and 1E are side views of the device 10A in the open state while holding an IOL; FIG. 1D is an isometric view of the device 10A in the closed state while holding the IOL; FIGS. 1F-1L illustrate examples of gradual/incremental displacements of the IOL holding portion of the device 10A along the axial, Z, direction.

Typically, the device 10A is implanted in the eye capsule, when in the closed state shown in FIGS. 1B and 1D. If a correction of the IOL position is warranted, the device 10A is activated to thereby move incrementally into one of the open states, such as shown in FIGS. 1F-1K.

As shown in FIG. 1A, the device 10A includes a first member 100A, a second member 200A, and a structure 300A connecting between the first and second members. In this non-limiting example, the device includes a locking mechanism 400A configured to control the structure 300A, e.g. its three dimensional shape, as will be described below. The first member 100A is configured to be fixedly positioned inside the lens capsule. The second member 200A is configured to hold the IOL fixedly. In this example, the second member 200A includes a cavity 202A configured to receive the IOL.

In the present and other following examples, the device substantially traces a circular shape, the first member 100A substantially forms an outer ring and the second member 200A substantially forms an inner ring that resides inside the outer ring of the first member in the closed state of the device. It is noted, however, that the first and second members, each or both, can take other shapes, and they can have, inter alia, open shapes and not necessarily closed shapes as in the present example. For example, the first and/or the second members may be configured as open shapes, e.g. open arcs.

The devices of the invention, while holding the IOL, are typically implanted in the anatomical lens capsule compartment, or in the anatomical sulcus in case the lens capsule is damaged/ruptured. Usually, the implantation of the IOL in the human eye is supported by one or more haptics that are attached to the device and that can anchor the device and the IOL to the implantation site. In some embodiments, the first member includes at least two integral haptics (not shown) on the outer side thereof. In some other embodiments, the first member includes at least two attachment portions (not shown), on the outer side thereof, configured for attaching thereto two corresponding haptics. The haptics may be adjusted to the specific implantation anatomical site.

The dimensions of the devices of the present invention are selected to enable secure holding of lenses, including off-the-shelf lenses, and to insure secure implantation and effective displacement of the IOL after the implantation. The devices are configured to hold the IOL in a permanent position until the device is activated to displace the IOL.

The structure 300A and the locking mechanism 400A form a movement mechanism/assembly responsible for displacing the second member 200A, and the IOL held thereby, relative to the first member 100A, relative to/along the Z direction.

The structure 300A is a bendable structure that is fixedly/permanently attached at a first end thereof (e.g. 300AF) to the first member 100A and at a second end thereof (e.g. 300AS) to the second member 200A. The bendable structure 300A is configured to be remotely controllably bended at various bending levels to thereby vary location of a periphery of the IOL, when the IOL is attached to the second member, along the IOL optical axis.

In some embodiments, the bendable structure is made, partially or fully, from a shape memory material and/or super elastic material (e.g., Nitinol, Bi-Metal, as will be further described below), treated such that default shape of the bendable structure is in its fully open state, as shown in FIG. 1A for example. The locking mechanism is operable to vary position and/or three-dimensional shape of the bendable structure between a fully closed state, as shown in FIG. 1B for example, and the fully open state as shown for example in FIG. 1K.

By controlling the locking mechanism remotely, one or more portions of the bendable structure can be locked to or released from either the first member or the second member, thereby collapsing or extending at least a portion of the bendable structure along the Z direction and displacing the second member with respect to the first member, thereby varying location of at least a portion/periphery of the IOL, when attached to the second member, along the Z direction/IOL optical axis OA. In this non-limiting example, the locking mechanism 400A is controlled remotely to release one or more portions of the bendable structure 300A from the first member 100A, thereby extending at least a portion of the bendable structure 300A along the Z direction and displacing the second member 200A with respect to the first member 100A, thereby varying location of at least a portion/periphery of the IOL, when attached to the second member, along the Z direction/IOL optical axis OA.

In some embodiments, the locking mechanism is operable to at least partially lock or release the one or more portions of the bendable structure to/from either the first member or the second member, in a way such that first and second planes defined by the first and second members, respectively, remain parallel therebetween and perpendicular to the IOL optical axis during varying the location of the periphery of the IOL along the IOL optical axis. In the described example, as shown for example in FIGS. 1A and 1C, the locking mechanism 400A is operable to release one or more portions of the bendable structure 300A from the first member 100A such that the plane P1 defined by the first member 100A and the plane P2 defined by the second member 200A remain parallel therebetween and perpendicular to the IOL optical axis Z/OA during varying the location of the periphery of the IOL along the IOL optical axis.

In some embodiments, the locking mechanism is operable to at least partially lock or release the one or more portions of the bendable structure to/from either the first member or the second member, in a way such that a first plane defined by the first member becomes tilted with respect to a second plane defined by the second member during varying the location of the periphery of the IOL along the IOL optical axis. This is shown, for example in FIG. 1E, in which the plane P1 and the plane P2 are inclined/tilted with respect to each other. This can be achieved, for example, by asymmetrical activation of different portions of the locking mechanism, as will be also shown further below in FIG. 1L. It is understood that the device can be configured to enable tilting in X and/or Y directions.

In some embodiments, the bendable structure includes a plurality of elongated bendable members, each of the elongated bendable members having a first end permanently/fixedly attached to the first member and a second end permanently/fixedly attached to the second member. In this non-limiting example, the bendable structure includes four elongated bendable members, 300A1-300A4, distributed over the circular perimeters of the first and second members, and, as seen, each of the elongated bendable members is permanently connected at a first end thereof to the first member and at a second end thereof to the second member. As also shown, each elongated bendable member is a continuous single element.

In some embodiments, the locking mechanism includes a plurality of remotely controllable lockers, each of the lockers is configured to lock/release a portion of the bendable structure to/from either the first or second members. This is exemplified in the current example where the locking mechanism 400A includes twenty eight individually controllable lockers, such as the lockers 400A1 and 400A2 illustrated in FIG. 1A1. Further details relating to the lockers are mentioned further below.

In some embodiments, the locking mechanism includes at least two groups of remotely controllable lockers and the bendable structure includes respective at least two elongated bendable members. In this non-limiting example, there are four groups of lockers 400AA-400AD, and the four elongated bendable members 300A1-300A4 respectively (as exemplified in FIG. 1K). Each of the groups of controllable lockers contains an array of seven individual lockers operable to lock or release the respective elongated bendable member to/from the first member 100A. The individual lockers of the locker array lock respective portions of the elongated bendable member and are sequentially activated to release respective portions of the elongated bendable member. For example, the lockers 400A1, 400A2 and 400A3, illustrated in FIG. 1B1, are activated in this order, resulting in the open configuration shown in FIG. 1A1, to release respective portions of the elongated bendable member 300A1.

Symmetrical activation of the individual controllable lockers belonging to the different groups of the controllable lockers causes varying the location of the periphery of the IOL in a perpendicular direction to the IOL optical axis, in other words keeping the planes P1 and P2, defined by the first and second members respectively, parallel. This is exemplified in FIGS. 1F-1K. For example, as illustrated in FIG. 1F, the first locker in the locker array of each group of the four groups is activated to release the first portion of the respective elongated member. As illustrated in FIG. 1G, the second locker of each group is activated to release the second portion of the respective elongated member. In FIGS. 1H-1K, the third, fourth, fifth and sixth locker of each group is respectively activated to release the third, fourth, fifth and sixth portion of the respective elongated member. Size, distance between and number of the lockers all play role in the displacement distance along the IOL optical axis, for a given length and default shape of the elongated bendable member. As such, it is appreciated that a constant distance between each two adjacent lockers will yield a constant displacement distance, whereas varying the distance between the lockers in the array will yield varied displacement distances. This is true in all the embodiments herein that employ the locking mechanism in the form of one or more locker arrays.

On the other side, asymmetrical activation of the individual controllable lockers belonging to the different groups of the controllable lockers causes varying the location of the periphery of the IOL in a tilted direction to the IOL optical axis. This is the case shown in FIG. 1L for example, where the groups 400AB and 400AC are activated one time more that the groups 400AA and 400AD. Control on the tilt angle is achieved based for example on size, distance between and number of the lockers, for a given length and default shape of the elongated bendable member.

It is appreciated that the device, the bendable structure and the locking mechanism can be designed to enable displacement of the second member, and the IOL attached thereto, in the anterior direction (towards the cornea) only, or the posterior direction (towards the retina) only, or in both of these directions.

According to the invention, the IOL displacement is done remotely when one or more activable portions of the device are exposed to energy from an external energy source.

In some embodiments, the locking mechanism, specifically the controllable lockers, is/are made from a shape memory material (e.g., Nitinol, as will be further described below) treated such that at body temperature the locker is in a closed state locking the respective portion of the bendable structure and such that when heated to a predefined temperature, by absorbing energy from the remote energy source, the locker transitions into open state releasing the respective portion of the bendable structure. The two spatial configurations of the locker are shown in FIG. 1B1, illustrating the closed locking state, and in FIG. 1A1, illustrating the open unlocking state.

As described above, the bendable structure and/or the locking mechanism may include shape memory materials/super-elastic materials which enable the device, or major portions thereof, to be foldable under application of certain amount of external forces and to return to their original shape without deformation once the external forces are removed.

In some embodiments, the shape-memory material is a specifically designed Nitinol (Nickel Titanium alloy), chosen for its biocompatibility and design flexibility. In some embodiments, the super-elastic material is a specifically designed Nitinol or a bi-metal.

Nitinol can be designed to be a super-elastic material at a specific temperature range, and a shape-memory material at a specific temperature range. In general, Nitinol is configured to change its structure from martensitic phase to austenitic phase under gradient of a few Celsius degrees. The gradient of temperatures and the phases transformation temperatures can be programmed according to the requirements. For example, the Nitinol Alloy can be designed such that up to about 40° C. (close to the body temperature) it is in martensite phase being fictile and can be shaped to a desired shape under external forces, e.g. forming the bendable structure. On the other side, raising the temperature to about 60° C. causes phase transition into austenite phase where Nitinol changes its shape (deforms) to take a shape saved in its “memory” even while under certain amount of external forces, e.g. forming the open unlocking state of the lockers in the locking mechanism. Once the temperature returns to about 40° C., the Nitinol returns back to its fictile state and can be reshaped as desired, providing the closed locking state of the lockers. The bendable structure, the first member, the second member and the locking mechanism can be designed to be super-elastic around room temperature and around body temperature, such that they can be folded while being inserted into the lens capsule. For example, the device can be configured to be foldable such that it can be passed through a cross-section of about 2.54 mm² (equivalent to 1.8 mm circular diameter, though the cross-section can take an oval-like shape).

The remote energy source is configured and operable to provide activation energy to the plurality of lockers in the locking mechanism. Typically, each locker is activated individually. In some embodiments, the remote energy source requires direct/uninterrupted line of sight/route between the remote energy source and the locker being remotely activated, while in some other embodiments, there is no such requirement and the activation can be achieved without direct line of sight. In some embodiments, the remote energy source is configured and operable to provide the activation energy in the form of heat. This is particularly important in case the actuators are made from Nitinol, as mentioned above. In some embodiments, the remote energy source includes at least one radiating element operable to heat the lockers by irradiating them. In some embodiments, the remote energy source includes an electromagnetic radiation transmitter and the plurality of actuators include corresponding electromagnetic radiation receivers. In some embodiments, the remote energy source is a laser source. In some embodiments, the laser source is configured and operable to provide continuous laser radiation. In some embodiments, the laser source is configured and operable to provide a green spectrum of light (the so-called Argon laser). In some embodiments, the laser source is configured and operable to produce laser having the following parameters: laser power between 0.1-5 watt, laser pulse width between 200-1000 ms.

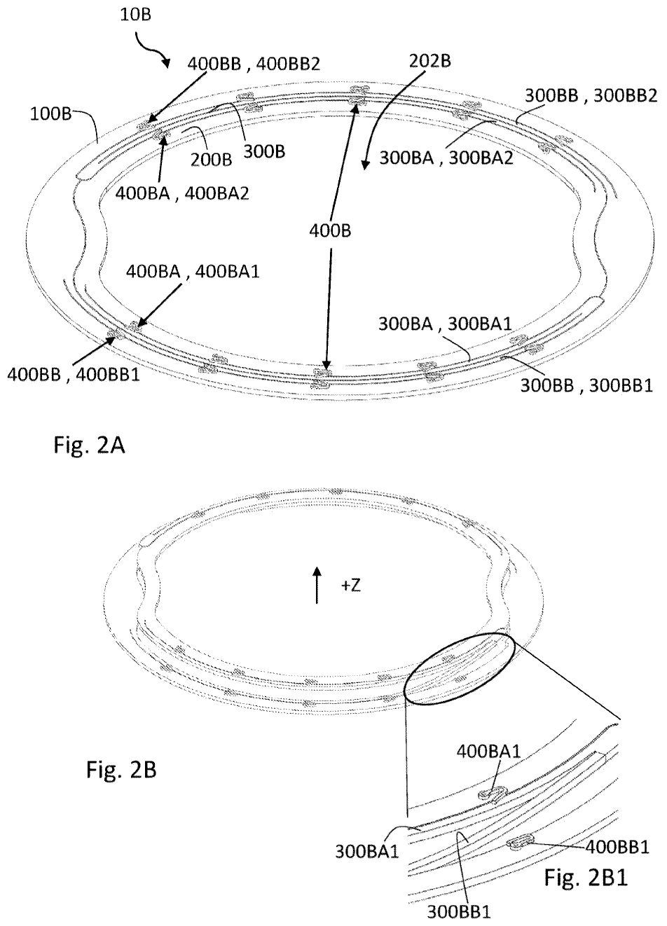

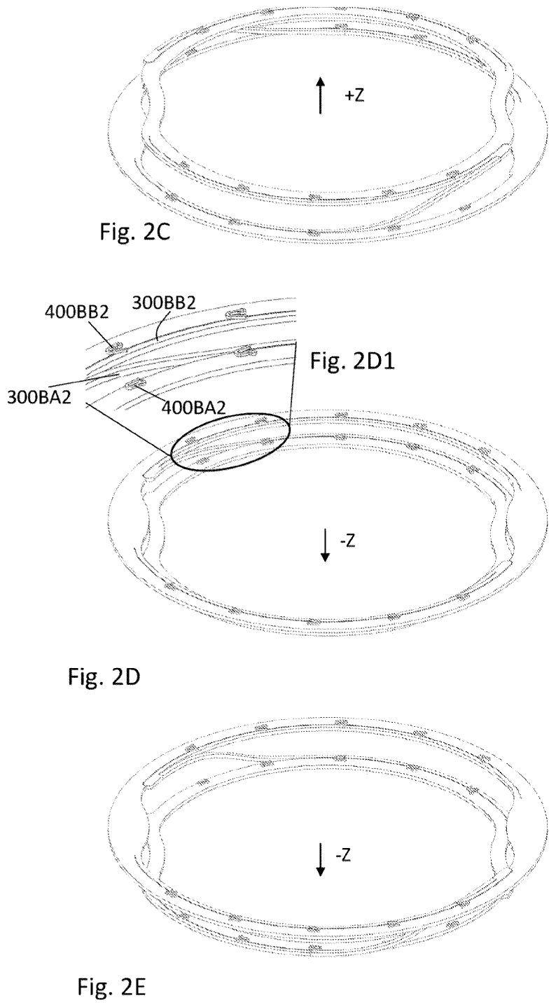

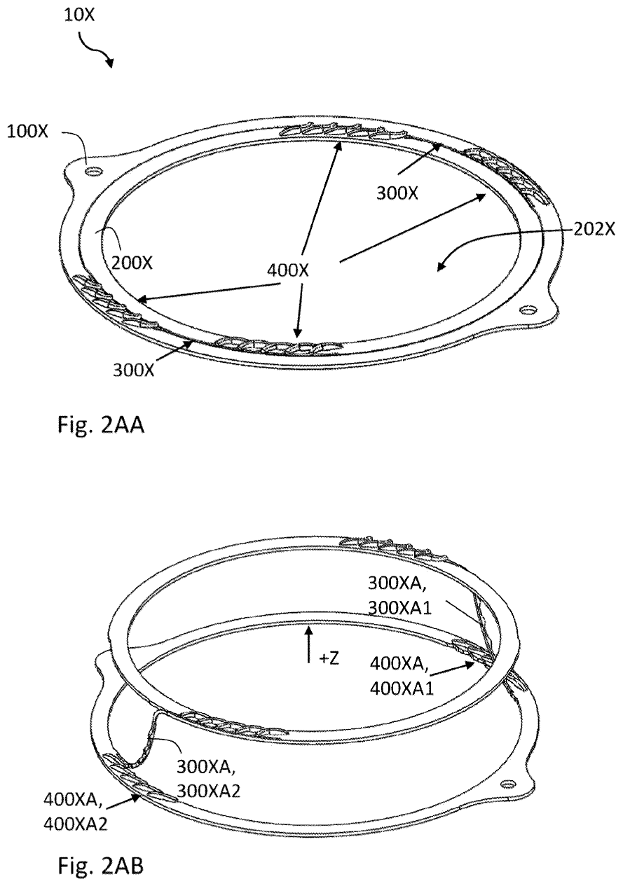

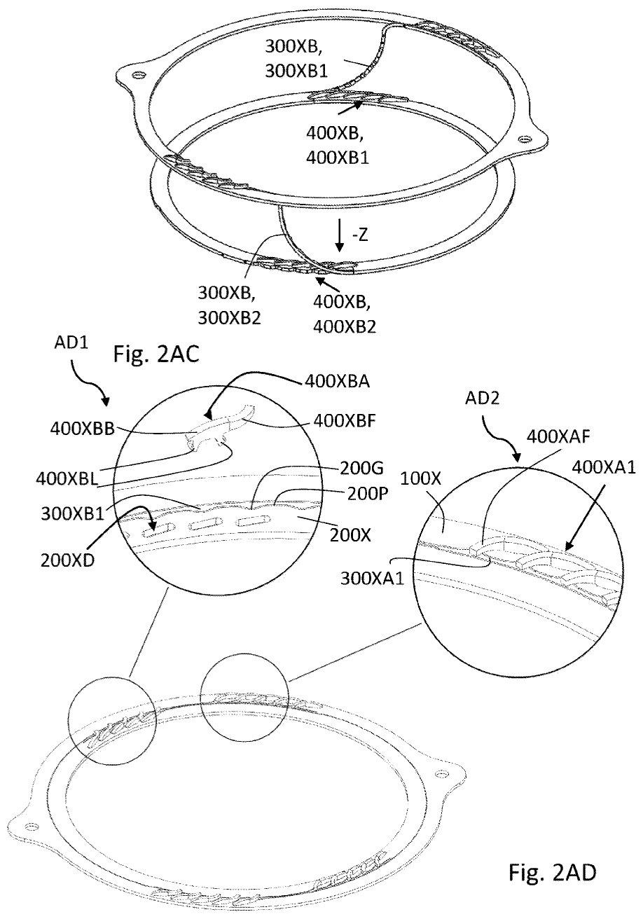

Reference is made to FIGS. 2A-2E illustrating another non-limiting example of a device 10B incorporating features of the present invention. As appreciated, the device 10A is configured for unidirectional displacement of the IOL along Z/OA direction. In contrast, as will be described below, the device 10B is configured for bidirectional displacement of the IOL along Z/OA direction.

FIG. 2A is a perspective upper view of the device 10B in its closed state ready for implantation inside the eye capsule together with IOL attached thereon; FIGS. 2B-2C illustrate two stages of displacement of the IOL along one Z/OA direction; and FIGS. 2D-2E illustrate two stages of displacement of the IOL along the opposite Z/OA direction.

As shown in FIG. 2A, the device 10B includes a first member 100B configured to be fixedly positioned inside the lens capsule, a second member 200B including a cavity 202B configured to hold the IOL fixedly, and a bendable structure 300B connecting between the first and second members. The device 10B also includes a locking mechanism 400B configured to control the structure 300B as will be described below.

The bendable structure 300B includes two branches 300BA and 300BB responsible for displacing the second member and the IOL in opposite Z directions. The first bendable structure branch 300BA includes two first elongated bendable members 300BA1 and 300BA2 responsible for displacing the IOL in a first Z direction, and the second bendable structure branch 300BB includes two second elongated bendable members 300BB1 and 300BB2 responsible for displacing the IOL in a second, opposite, Z direction. The movement in +Z and −Z directions is governed by the free unlocked and default shape of the associated bendable member.

The locking mechanism 400B includes two locker array branches 400BA and 400BB associated with the two bendable structure branches 300BA and 300BB respectively. The locker array branch 400BA is attached to the inner member 200B and the locker array branch 400BB is attached to the outer member 100B. Each of the locker array branches includes two locker arrays associated with the two elongated bendable members of each bendable structure branch. Each locker array of the two locker arrays in a locker array branch includes five individually controllable lockers. It is noted that a different number of lockers can exist in each locker array based on the specific application and need. As shown in FIGS. 2B and 2B1, for example, a first locker in the locker array 400BB1 is activated by the remote source energy such that it releases the elongated bendable member 300BB1. Concurrently, while not shown, to keep the first and second members in parallel and perpendicular to the OA direction, a first locker in the locker array 400BB2 is activated by the remote source energy such that it releases the elongated bendable member 300BB2. Accordingly, the second member and the IOL are displaced in the +Z direction.

FIG. 2C illustrates a second activation of the subsequent lockers in the arrays 400BB1 and 400BB2 to cause a second step displacement in the +Z direction by further releasing another portion of the elongated bendable members 300BB1 and 300BB2.

FIGS. 2D, 2D1 and 2E illustrate two sequential step displacements of the second member and the IOL in the −Z direction. In this case, the lockers in the locker arrays 400BA1 and 400BA2 are activated to release the elongated bendable members 300BA1 and 300BA2 respectively.

It is appreciated that the device 10B enables reversible displacement of the IOL. In other words, it is possible to reverse the direction of displacement and correct in the opposite direction if the displacement in a specific Z direction was oversized. So for example, if the IOL is displaced x steps in the +Z direction by activating the locker arrays 400BB1 and 400BB2, and there is a need to correct to (x−1) steps in the +Z direction, the locker arrays 400BA1 and 400BA2 are activated to displace the IOL one step in the −Z direction, giving an overall (x−1) step displacement in the +Z direction.

All the features described with respect to the device 10A are also applicable with respect to the device 10B, though not specifically described. For example, asymmetric activation of the lockers in the locker arrays 400BA1 and 400BA2 will cause a tilted displacement of the IOL along the IOL optical axis.

Reference is made to FIG. 2AA-2AD illustrating another non-limiting example of a device 10X incorporating features of the present invention. As appreciated, similar to the device 10B, the device 10X, as will be described below, is configured for bidirectional displacement of the IOL along Z/OA direction.

FIG. 2AA is an upper isometric view of the device 10X in its closed state ready for implantation inside the eye capsule together with an IOL attached thereon; FIG. 2AB-2AC illustrate displacement of the IOL along a first and second (opposite) Z/OA directions respectively; FIG. 2AD illustrates additional features of the devices of the present invention, exemplified on the device 10X.

As shown in FIG. 2AA, the device 10X includes a first (outer) member 100X configured to be fixedly positioned inside the lens capsule, a second (inner) member 200X including a cavity 202X configured to hold the IOL fixedly, and a bendable structure 300X connecting between the first and second members. The device 10X also includes a locking mechanism 400X configured to control the structure 300X as will be described below.

As can be seen in FIG. 2AB and 2AC, the bendable structure 300X includes two branches 300XA and 300XB responsible for displacing the second member and the IOL in opposite Z directions. The first bendable structure branch 300XA includes two first elongated bendable members 300XA1 and 300XA2 responsible for displacing the IOL in a first Z direction, and the second bendable structure branch 300XB includes two second elongated bendable members 300XB1 and 300XB2 responsible for displacing the IOL in a second, opposite, Z direction. The movement in +Z and −Z directions is governed by the free unlocked and default shape of the associated bendable member.

The locking mechanism 400X includes two locker array branches 400XA and 400XB associated with the two bendable structure branches 300XA and 300XB respectively. The locker array branch 400XA is attached to the outer member 100X and the locker array branch 400XB is attached to the inner member 200X. Each of the locker array branches includes two locker arrays associated with the two elongated bendable members of each bendable structure branch. For example, the locker array branch 400XB includes two locker arrays, 400XB1 and 400XB2, associated with the two elongated bendable members, 300XB1 and 300XB2, of the bendable structure branch 300XB. Each locker array of the two locker arrays in a locker array branch includes, in this non-limiting example, five individually controllable lockers. It is noted that a different number of lockers can exist in each locker array based on the specific application and need. Also noted, equal/different spacing between lockers achieves equal/different step (displacement) size. As explained above, to keep the first and second members 100X and 200X in parallel and perpendicular to the OA direction, identical number of lockers in the two locker arrays belonging to the same locker array branch are activated by the remote source energy such that the elongated bendable members of the same bendable structure branch are symmetrically released. Activating lockers in this way, moves the inner member in a stepped manner, with each individual locker activation, in +Z or −Z directions. At the same time, it is appreciated that asymmetrical activation of lockers belonging to the same locker array branch causes a tilt in the inner member 200X with respect to the outer member 100X. It can also be appreciated that asymmetrical or even symmetrical activation of lockers (e.g. the first lockers in the respective arrays) belonging to the two locker array branches may cause a tilt in the inner member 200X with respect to the outer member 100X.

It is appreciated that the device 10X enables reversible displacement of the IOL. In other words, it is possible to reverse the direction of displacement and correct in the opposite direction if the displacement in a specific Z direction was oversized. So for example, if the IOL is displaced x steps in the +Z direction by activating the locker arrays 400XA1 and 400XA2, and there is a need to correct to (x−2) steps in the +Z direction, the locker arrays 400XB1 and 400XB2 are activated to displace the IOL two steps in the −Z direction, giving an overall (x−2) step displacement in the +Z direction.

FIG. 2AD illustrates two additional features of the device 10X, which can also be implemented in all of the devices of the present invention. In the close-up view AD1, a locker 400XBA is shown. The locker 400XBA includes a back portion 400XBB configured to be mechanically attached to the inner member 200X in a reversible way that enables mechanical attachment and detachment of the locker. In this non-limiting example, the locker includes two protrusions/legs 400XBL that are bendable and insertable into a corresponding depression 200XD formed in the inner member. Once the legs are bended and inserted they open back to the default shape and are locked securely inside the depression at corresponding lock points. In other words, the locker is attached in a clipping mechanism. For example, as described above, the legs of the locker can be made from a flexible Nitinol (e.g., Nitinol in martensite phase at appropriate temperature range, e.g. including room temperature and body temperature). As appreciated, other non-permanent attachment mechanisms, e.g. based on male-female attachments, are also possible. In comparison to other attachment mechanisms, specifically permanent mechanisms such as welding or laser welding or gluing or soldering or a combination thereof, that can be applied in some embodiments herein, this mechanical attachment provides secure, accurate, repeatable and controllable positioning and alignment of the locker. It is appreciated that since the legs are fixed inside the depression (while also being made from Nitinol), only a front portion 400XBF of the locker is activated by the external energy source to deform and release the associated portion of the bendable member.

Another feature is also shown in view AD1. The inner or outer member and the bendable member associated with each locker array are correspondingly shaped in matching groove-protrusion (or valley-hill) fashion. Each protrusion in the outer/inner member is aligned with a matching groove in the bendable member. In this example, the inner member 200X and the bendable member 300XB1 associated with the locker array 400XB1 are correspondingly shaped in matching groove-protrusion fashion. As shown in close-up view AD2, each locker's front portion 400XAF of the lockers in the locker array 400XA1 is aligned, when in the closed locking state, with a protrusion formed in the bendable member 300XA1 and a corresponding groove formed in the outer member 100X. In the open unlocking state, the front portion of the locker will be aligned with the protrusions of the inner or outer member, as the case may be. These specific shaping and alignment enable secure locking of the bendable member by the lockers in view of the miniature size of the lockers and the bendable member, thus facilitating producing the tiny device of the invention while maximizing its functionality. In addition, this enables to minimize the needed movement of the locker between the locking and unlocking states, therefore optimizing energy usage and/or amount of shape memory material usage.

Other features described with respect to the devices 10A and 10B are also applicable with respect to the device 10X, though not specifically described.

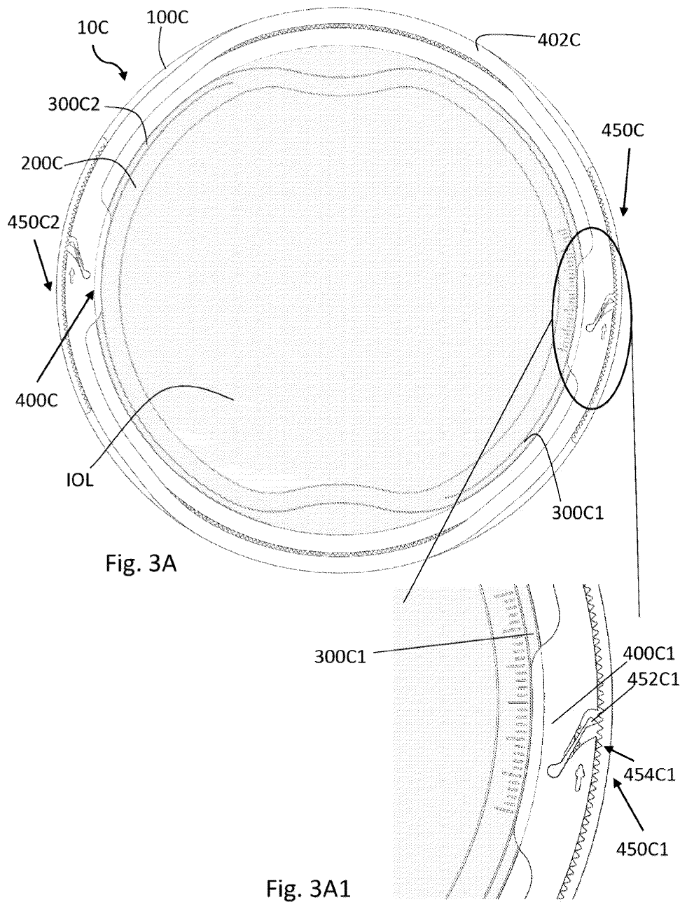

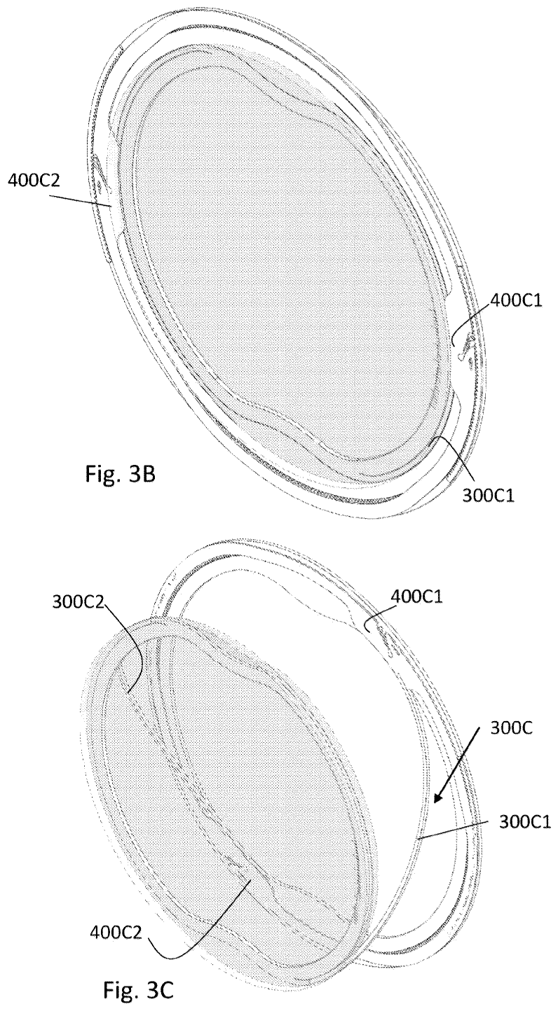

Reference is made to FIGS. 3A-3C illustrating another non-limiting example of a device 10C incorporating features of the present invention. FIG. 3A is a front view of the device 10C; FIG. 3A1 is a close-up view of a portion of the device 10C; FIG. 3B illustrates the device 10C when holding the IOL with zero displacement in Z direction; and FIG. 3C illustrates the device 10C when holding the IOL with a displacement in Z direction.

As shown in FIGS. 3A and 3A1, the device 10C includes a first member 100C configured to be fixedly positioned inside the lens capsule, a second member 200C configured to hold the IOL, and a bendable structure 300C (FIG. 3C) interconnected between the first and second members.

In this non-limiting example, the displacement of a periphery of the IOL along the z direction is achieved by a locking mechanism 400C that at least partially releases or locks the bendable structure to the first member 100C, utilizing a rotational movement of the locking mechanism 400C that releases or locks the bendable structure 300C when the locking mechanism is rotated clockwise or counterclockwise.

Specifically, as shown in the figures, the locking mechanism 400C includes a third member 402C adjacent, specifically on top of, the first member 100C such that the bendable structure 300C is restricted/enclosed between the first and third members. The locking mechanism 400C includes one or more lockers, e.g. 400C1 and 400C2, that lock or release one or more portions of the bendable structure, such as the elongated bendable members 300C1 and 300C2, when the third member 402C is rotated with respect to the first member 100C, varying the location of the peripheral of the IOL along the Z/OA direction. As shown, the lockers 400C1 and 400C2 can be configured as local protrusions along the inner perimeter of the third member 402C.

FIG. 3B illustrates the closed initial state, when the bendable structure is locked to the first member, and FIG. 3C illustrates counterclockwise rotation of the third member 402C and the lockers 400C1 and 400C2 to gradually release the elongated bendable members 300C1 and 300C2 respectively. It is appreciated that clockwise rotation of the lockers 400C1 and 400C2 will gradually lock the elngated bendable members 300C1 and 300C2 back to the first member thereby decreasing the displacement of the periphery of the IOL along the optical axis. It is also appreciated that this example illustrates symmetrical displacement of the IOL such that the IOL is kept perpindicular to the optical axis with no tilt.

The rotation of the third member 402C that carries the lockers 400C1 and 400C2 is achieved by the help of a rotation mechanism 450C, included in the locking mechanism, that can be remotely controlled with the remote energy source. As shown in FIGS. 3A and 3A1, the rotation mechanism 450C includes two rotation sub-mechanisms, a first sub-mechanism 450C1 responsible for rotating the third member 402C counterclockwise, and a second sub-mechanism 450C2 responsible for rotating the third member 402C clockwise.

The rotation mechanism 450C can be implemented in a variety of ways. In this non-limiting example, the rotation mechanism includes an actuator, such as 452C1 of sub-mechanism 450C1, having a fixed connection with the third member 402C, the actuator is configured to be remotely activated by the remote energy source to engage with an interaction region 454C1 having a fixed connection with the first member 100C, such that the engagement causes the rotation movement of the third member 402C.

For example, as shown, the interaction region 454C1 includes a series of teeth/ridges and valleys with which the actuator 452C1 interacts. The interaction region can extend over a predefined distance defined by the length of the elongated bendable member. Also, the tooth size defines the step size of the rotational movement that occurs and the corresponding released or locked portion of the bendable structure. More specifically, the actuator 452C1 is made from a shape memory material, e.g. Nitinol, that is configured, when heated by energy absorbed from the remote energy source, to move from a disengaged position to an engaged position in which the actuator pushes against a tooth of the interaction region and causes the third member 402C to rotate. Similar configurations of the rotation mechanism are described in WO2018229766A1, assigned to the assignee of the present invention.

It is appreciated that the described rotation mechanism(s) can be incorporated in any of the devices described above, in addition to the bendable structure, to enable independent displacement of the IOL along theta direction, i.e. to enable rotation of the IOL around the optical axis, such that two independent displacements can be obtained, both along and around the optical axis.

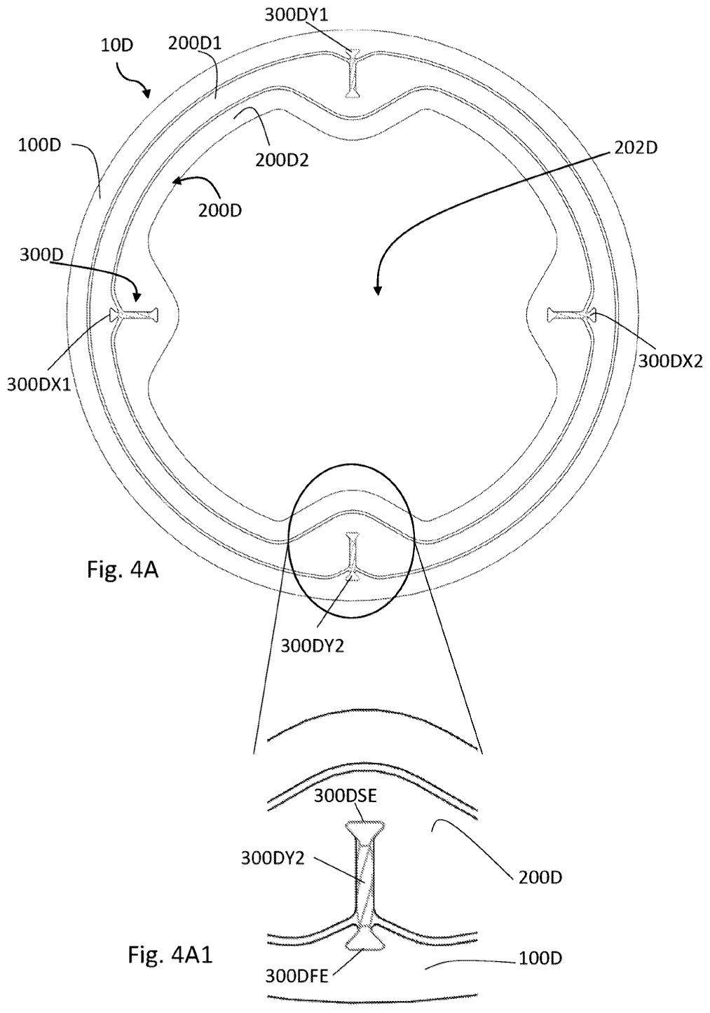

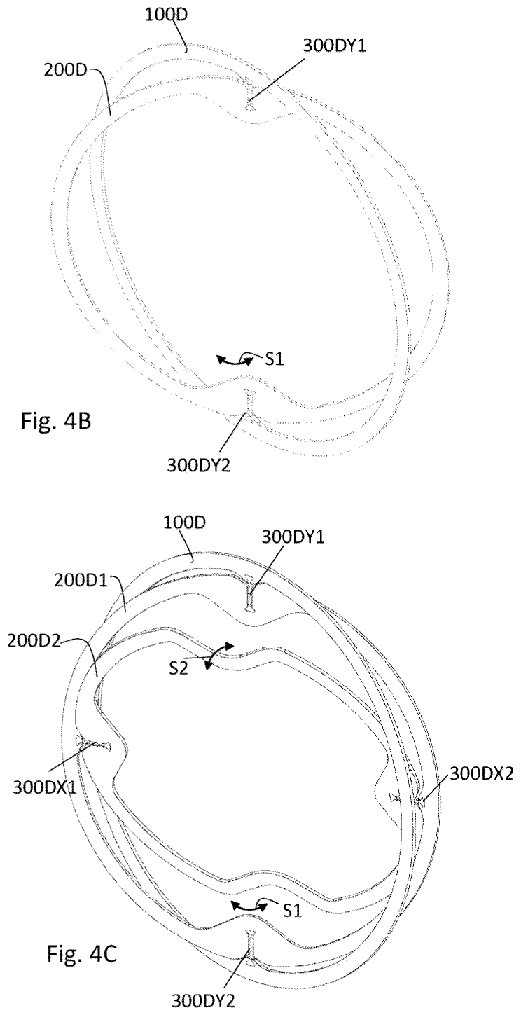

Reference is made to FIGS. 4A-4C illustrating another non-limiting example of a device 10D incorporating features of the present invention. FIG. 4A is a front view of the device 10D; FIG. 4A1 is a close-up view of the bendable structure in device 10D; FIG. 4B illustrates the tilting of the IOL around a first axis; and FIG. 4C illustrates the tilting of the IOL around first second axes.

The device 10D illustrates a pure tilting mechanism. However, it is appreciated that it can also be integrated with one of the Z direction displacement mechanisms described above.

As shown in FIG. 4A, the device 10D includes a first member 100D configured to be fixedly positioned inside the lens capsule, a second member 200D including a cavity 202D configured to hold the IOL, and a bendable structure 300D connecting between the first and second members.

The bendable structure 300D includes a plurality of elongated bendable members, specifically two bendable members 300DY1 and 300DY2 having a first end, 300DFE in FIG. 4A1, permanently/fixedly attached to the first member 100D and a second end 300DSE permanently/fixedly attached to the second member 200D, specifically to a first portion 200D1 of the second member. The pair of the two bendable members 300DY1 and 300DY2 are located on opposite sides with respect to the second member, and the IOL when attached. Each bendable member is twistable around a longitudinal axis thereof. As shown, in the default starting configuration, when there is no displacement of a periphery of the IOL along Z axis, each bendable member is preloaded by being twisted around its longitudinal axis. The bendable members are made at least partially from a shape memory material that can be controlled to alter its shape when absorbing energy from the remote energy source. The first bendable member, e.g. 300DY1, can be twisted with certain amount of twists in a clockwise direction, such that each activation causes the relief of one twist which then translates into a defined degree of IOL tilt into a counterclockwise direction. The second bendable member 300DY2 can be twisted with certain amount of twists in a counterclockwise direction, such that each activation causes the relief of one twist which then translates into a defined degree of IOL tilt into a clockwise direction. This is illustrated in FIG. 4B. FIG. 4B1 includes two bendable members 300DY1 and 300DY2 that enable rotation of a single second member 200D around one axis S1.

In some embodiments, such as in this non-limiting example, the second member can include two portions that can be rotated with respect to each other, thereby adding another dimension of tilting directions of the IOL. As shown, in FIG. 4C, the second member 200D includes a first portion 200D1 that can be swiveled in S1 directions, utilizing the first pair of bendable members 300DY1 and 300DY2, and a second portion 200D2 that can be swiveled in S2 directions, utilizing the second pair of bendable members 300DX1 and 300DX2.

In some embodiments, the device of the invention, such as the devices described above, is enclosed within an enclosure that helps to protect and shield the movable and functional parts of the device, e.g. the bendable structure and the locking mechanism, and prevents risks of clogging and immobilization due to interaction of the movable and functional parts with physiological medium inside the body, e.g. inside the eye capsule that receives the device holding the IOL.

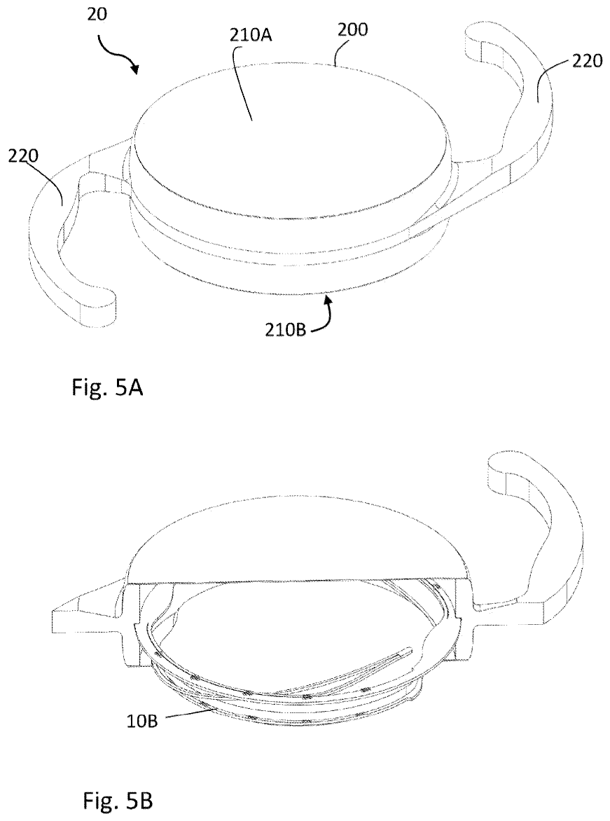

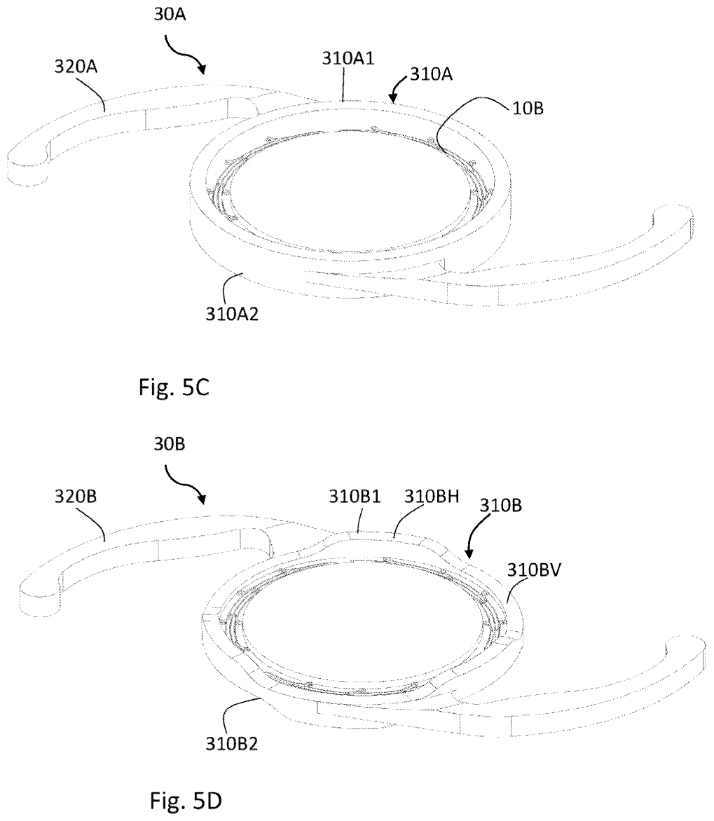

Reference is made to FIGS. 5A-5B illustrating a first non-limiting example of a device 20 incorporating features of the present invention. As shown, the device 20 includes an enclosure/cover/shell 200 that is transparent for the incoming light, at least on sides/surfaces 210A and 210B interfacing with the optical path of light entering the eye, and is aimed to house and protect any one of the devices described above. Specifically, the device 20 shields the movable and functional parts of the devices described above, e.g. the bendable structure and the locking mechanism, and prevents the risks of clogging and immobilization due to interaction of the movable and functional parts with physiological medium inside the body. In this non-limiting example, the device 20 is illustrated with the device 10B enclosed inside the enclosure 200, as shown in FIG. 5B with a portion of the device 20 removed. Also, the device 20 is illustrated with two peripheral haptics 220 that aid in implanting the device 20 with the enclosed device 10B in the eye capsule. It is appreciated that the device 20 can be adjusted to the enclosed device and can be configured in a variety of ways.

Reference is made to FIGS. 5C-5D, illustrating a second and a third non-limiting examples of devices 30A and 30B incorporating features of the present invention. As shown in FIG. 5C, the device 30A includes a housing 310A that includes walls 310A1 and 310A2 extending along the axis Z above and below the device 10B and does not include transparent surfaces/covers interfacing with the optical path of light entering the eye. As shown in FIG. 5D, the device 30B includes a housing 310B that includes walls 310B1 and 310B2 extending along the axis Z above and below the device 10B and does not include transparent surfaces/covers interfacing with the optical path of light entering the eye. Specifically, the devices 30A and 30B shield the movable and functional parts of the devices described above, e.g. the bendable structure and the locking mechanism, by preventing collapse of the eye capsule walls onto the movable/functional parts of the device 10B, thereby preventing the risks of clogging and immobilization of the movable and functional parts. In the specific example of FIG. 5D, the walls 310B1 and 310B2 of the devices are structured in a wave-like structure, e.g. a hill 310BH and valley 310BV fashion that aids in the foldability of the device 30B to facilitate its insertion through as small as possible slit in the eye wall and into the eye capsule. The device 30B includes three hills (ridges, protrusions) and three valleys on each wall side. Each hill on the wall 310B1 is faced with a valley on the wall 310B2. In these non-limiting examples, the devices 30A and 30B are illustrated with two peripheral haptics 320A and 320B respectively that aid in implanting the devices 30A and 30B with the enclosed device 10B in the eye capsule. It is appreciated that the devices 30A and 30B can be adjusted to the enclosed device and can be configured in a variety of ways.

Accordingly, it is appreciated that the present invention provides a powerful technique for controllable and precise remote displacement of an IOL along its optical axis, after the IOL has been implanted, to adjust the IOL position and enable it to function properly.

1. A device configured to be implanted in a lens capsule of a human eye and securely hold an intraocular lens (IOL), and operable to displace the IOL relative to an optical axis of the IOL, the device comprising:
a first member configured to be fixedly positioned inside the lens capsule;
a second member to which the IOL is fixedly attachable;
a bendable structure permanently attached at a first end thereof to the first member and at a second end thereof to the second member; the bendable structure being configured to be remotely controllably bended at various bending levels; and
a locking mechanism locking the bendable structure to the first and second members, the locking mechanism configured to be remotely controllable and operable to at least partially release one or more portions of the bendable structure to/from either the first member or the second member, to thereby vary location of a periphery of the IOL, when the IOL is attached to the second member, along the IOL optical axis.

2. (canceled)

3. The device of claim 1, wherein said locking mechanism is operable to at least partially lock or release the one or more portions of the bendable structure to/from either the first member or the second member, in a way such that first and second planes defined by the first and second members respectively remain parallel therebetween and perpendicular to the IOL optical axis during varying the location of the periphery of the IOL along the IOL optical axis.

4. The device of claim 1, wherein said locking mechanism is operable to at least partially lock or release the one or more portions of the bendable structure to/from either the first member or the second member, in a way such that a first plane defined by the first member becomes tilted with respect to a second plane defined by the second member during varying the location of the periphery of the IOL along the IOL optical axis.

5. The device of claim 1, wherein said locking mechanism comprises a plurality of remotely controllable lockers, each of the lockers being configured to lock and release a portion of the bendable structure to/from either the first or second members.

6. The device of claim 1, wherein said bendable structure comprises a plurality of elongated bendable members, each of the elongated bendable members having a first end permanently attached to the first member and a second end permanently attached to the second member.

7. The device of claim 1, wherein:
said locking mechanism comprises at least two groups of remotely controllable lockers and said bendable structure comprises respective at least two elongated bendable members, each of the groups of controllable lockers being operable to lock or release the respective elongated bendable member to/from either the first or second members, each of the groups of controllable lockers comprises a plurality of controllable lockers arranged in a locker array such that sequential activation of the lockers in the locker array locks or releases respective portions of the elongated bendable member.

8. The device of claim 7, wherein:
symmetrical activation of the individual controllable lockers belonging to the different groups of the controllable lockers causes varying the location of the periphery of the IOL in a perpendicular direction to the IOL optical axis;
asymmetrical activation of the individual controllable lockers belonging to the different groups of the controllable lockers causes varying the location of the periphery of the IOL in a tilted direction to the IOL optical axis.

9. (canceled)

10. The device of claim 1, wherein said locking mechanism comprises a third member adjacent to the first member such that the bendable structure is enclosed between the first and third members, rotating the third member with respect to the first member locks or releases the one or more portions of the bendable structure to/from the first member, and varies the location of the peripheral of the IOL along the IOL optical axis.

11. The device of claim 1, wherein said bendable structure comprises a plurality of elongated bendable members, at least some of the elongated bendable members having a first end permanently attached to the first member and at least some of the elongated members having a second end permanently attached to the second member, each one of the plurality of elongated bendable members is twistable around a longitudinal axis thereof, the elongated bendable members being arranged in one or more pairs having the two elongated bendable members of the pair located on opposite sides with respect to the second member, remotely controlling the twist degree of the two elongated bendable members of the pair causes tilting the IOL, when attached to the second member, around an axis passing through the two elongated bendable members of the pair.

12. The device of claim 1, configured and operable to displace the IOL, when attached to the second member, in one or more of the following manners:
in at least one of anterior or posterior direction relative to the first member;
at least partially in a reversible manner.

13. (canceled)

14. The device of claim 1, wherein said bendable structure is made from a shape memory material treated such that default shape of the bendable structure is in its fully open state, wherein said locking mechanism is operable to vary position of the bendable structure between a fully closed state and the fully open state.

15. The device of claim 5, wherein each of the controllable lockers is made from a shape memory material treated such that at body temperature the locker is in a closed state locking the respective portion of the bendable structure and such that when heated to a predefined temperature, by absorbing energy from a remote energy source, the locker transitions into open state releasing the respective portion of the bendable structure.

16. The device of claim 11, wherein said bendable structure is made from a shape memory material treated such that at body temperature a default shape of each elongated bendable member is in its fully twisted state and such that when heated to a predefined temperature, by absorbing energy from a remote energy source, the elongated bendable member transitions into a less twisted state.

17. The device of claim 14, wherein said shape memory material comprises nitinol.

18. The device of claim 1, wherein said device is foldable such that it can be passed through a cross-section of about 2.54 mm² or about 1.8 mm circular diameter.

19. The device of claim 5, wherein said lockers are configured to be attachable to corresponding points on the first or second members, mechanically or by laser welding or gluing or a combination thereof.

20. The device of claim 19, wherein said lockers are attachable reversibly mechanically to the first or second members by clipping or male-female setup.

21. The device of claim 6, wherein said bendable members include, at a side thereof facing the first or second member, sequential grooves and protrusions matching sequential protrusions and grooves at the opposite side of the first or second member, the locking mechanism comprises a plurality of remotely controllable lockers, each of the lockers being configured to lock and release a portion of the bendable members to/from the first or second members, the lockers being aligned in their locking state with the protrusions of the bendable members and in their unlocking state with the protrusions of the first or second members.

22. An IOL adjustment system comprising:
the device of claim 1; and
a remote energy source configured and operable to provide said energy to heat one or more portions of the locking mechanism to cause the release of one or more portions of the bendable structure to/from either the first member or the second member.

23. The system according to claim 22, wherein said remote energy source comprises one of the following:
a radiating element;
a laser source:
a laser source configured and operable to provide continuous laser radiation;
a laser source configured as an Argon laser source operable to provide light of a green spectrum;
a laser source configured and operable to provide laser power between 0.1-5 watt, and laser pulse width between 200-1000 ms; or
an electromagnetic radiation transmitter and said one or more portions of the device comprise respective electromagnetic radiation receivers.

24. (canceled)

25. (canceled)

26. (canceled)

27. (canceled)

28. (canceled)
